(19) United States
(12) Patent Application Publication
(10) Pub. No.: US 2012/0046576 A1
(43) Pub. Date: Feb. 23, 2012

(54) Mid-face aesthetic scale and related methods

(75) Inventors: Xiaoming Lin, Cave Creek, AZ (US); David Morgan, Phoenix, AZ (US); Ira Lawrence, Chicago, IL (US); Mitch Wortzman, Scottsdale, AZ (US)

(73) Assignee: Medicis Pharmaceutical Corporation, Scottsdale, AZ (US)

(21) Appl. No.: 13/199,113

(22) Filed: Aug. 19, 2011

Related U.S. Application Data
(60) Provisional application No. 61/401,862, filed on Aug. 19, 2010.

(51) Int. Cl. A61B 5/103

(52) U.S. Cl. 600/587

(57) ABSTRACT

This disclosure relates to mid-face aesthetic scales and related methods for evaluating fullness in a mid-face area of a human patient's face. In exemplary embodiments, the mid-face area is defined as medial to the maxillary prominence, lateral to the medial canthus, inferior to the maxillary prominence, superior to the plane of nasal alae, and lateral to the nose. An exemplary method for evaluating fullness in a mid-face area of a human patient's face comprises visually comparing a plurality of views of the patient's mid-face against a mid-face scale, and assigning a grade from the scale to the patient's mid-face based on the visual comparison.

### CROSS-REFERENCE TO RELATED APPLICATIONS

This Non-Provisional Patent Application claims priority to and the benefit of Provisional Patent Application Ser. No. 61/401,862, entitled MID-FACE AESTHETIC SCALE AND RELATED METHODS and filed Aug. 19, 2010, which is incorporated by reference herein in its entirety.

### FIELD OF INVENTION

This disclosure generally relates to dermal filler regimens or other facial modification treatments and a scale for evaluating the fullness of the mid-face.

### BACKGROUND OF THE INVENTION

The facial stigmas of the aging process are caused by a combination of internal (e.g., aging and genetic) and external factors (e.g., exposure to sun and pollution). Together, they result in the diminished production of collagen, which is the main supporting protein of the skin, as well as a breakdown of elastin fibers, which gives the skin its flexibility. As individuals age, the bony skeleton and soft tissues of the face lose volume, drop and shrink to produce a wider orbital aperture and less anterior projection. This decreases the overall projection of the cheek and diminishes bony support for the overlying soft tissue structures. This aging process results in drooping eyes and tear trough deformity, lateral eyebrow ptosis, malar descent, a heavy jaw line and hypertonic contractions of the depressor muscles.

In addition to countering the aging process, the desire to undergo cosmetic treatments may arise from other factors, including general dissatisfaction with appearance and trauma. Dermal fillers are non-surgical cosmetic treatments that are used to give a more youthful appearance by restoring facial volume or fullness. They work by reducing or eliminating lines, wrinkles and folds in the skin. A major advantage of these treatments is that the effect is often seen immediately or shortly after treatment. There are various kinds of safe, natural and synthetic materials used as dermal fillers, including porcine and bovine collagen, calcium hydroxylapatite and hyaluronic acid (HA) such as Perlane® or Perlane-L®.

Mid-face augmentation is performed in clinical settings to achieve fullness in the malar area. Communicating the treatment goal with patients and measuring the treatment success of mid-face augmentation however, presents considerable difficulties. Previously known scales rely on computerized methods, are too complicated, do not use “real” facial images, and do not account for ethnic, age and gender variability, etc. Computer-morphed images are not “lifelike” and do not present the real life results after mid-face augmentation. Moreover, previously known scales do not define the “mid-face” optimally, while others fail to define the “mid-face” apart from the whole face. In short, there are no accepted, clinically validated scales, to measure the treatment success of mid-face augmentation. Accordingly, previously known scales have not proven to be clinically meaningful.

Thus, there exists a need in the art to have a validated mid-face scale that medical professionals can use to communicate the treatment goal with patients as well as to measure the treatment effect of mid-face augmentation, which scale could also be used as the primary efficacy measurement for mid-face augmentation in clinical studies. This disclosure addresses this need.

### SUMMARY OF THE INVENTION

This disclosure relates to mid-face aesthetic scales and related methods for evaluating fullness in a mid-face area of a human patient's face.

In an embodiment, this disclosure comprises providing a scale usable by medical professionals for assessing the fullness of the mid-face region, including use as a means for baseline assessment of the mid-face region and/or for assessment of the post-treatment effectiveness of dermal filler regimens or other facial modification treatments for improving the aesthetic appearance of the mid-face region.

In an embodiment, a scale is useful as the primary efficacy measurement for mid-face augmentation in clinical studies, for example, by researchers or administrative or regulatory bodies such as the United States FDA or other government authorities.

In an embodiment, a scale is useful as a communication tool with patients to understand baseline, deficits, and possible and realized improvements associated with a dermal filler regimen or other facial modification treatment.

An exemplary method for evaluating fullness in a mid-face area of a human patient's face comprises visually comparing a plurality of views of the patient's mid-face against a mid-face scale, and assigning a grade from the scale to the patient's mid-face based on the visual comparison. An exemplary method may precede and/or follow a dermal filler regimen, a mid-face augmentation, or any other mid-face modification treatment (e.g., implants, surgical procedures, etc.).

Exemplary embodiments include a method of assessing the effectiveness of a dermal filler regimen on a mid-face area of a human patient's face comprising evaluating the mid-face area before delivery of the dermal filler and classifying it on a scale as disclosed above, delivering to the mid-face area an amount of the dermal filler, and assessing whether the dermal filler was effective by re-evaluating the mid-face area after delivery of the dermal filler.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Exemplary embodiments will be described in conjunction with the accompanying drawing figures in which like numerals denote like elements and:

FIG. 1 illustrates the area defined by the inventors as the “mid-face,” in accordance with exemplary embodiments of this disclosure;

FIG. 2 consists of photographs of reference subjects depicting a grade 1 fullness level, in accordance with an exemplary mid-face scale of this disclosure;

FIG. 3 consists of photographs of reference subjects depicting a grade 2 fullness level, in accordance with an exemplary mid-face scale of this disclosure;

FIG. 4 consists of photographs of reference subjects depicting a grade 3 fullness level, in accordance with an exemplary mid-face scale of this disclosure;

FIG. 5 consists of photographs of reference subjects depicting a grade 4 fullness level, in accordance with an exemplary mid-face scale of this disclosure; and

FIG. 6 illustrates a camera system and an auxiliary flash unit in accordance with an exemplary embodiment of this disclosure.

### DETAILED DESCRIPTION

The detailed description of exemplary embodiments herein shows exemplary embodiments by way of illustration and its best mode. While these exemplary embodiments are described in sufficient detail to enable those skilled in the art to practice the invention, it should be understood that other embodiments may be realized and that logical, chemical and mechanical changes may be made without departing from the spirit and scope of the invention. Thus, the detailed description herein is presented for purposes of illustration only and not of limitation. For example, the steps recited in any of the method or process descriptions may be executed in any order and are not necessarily limited to the order presented. Moreover, many of the functions or steps may be outsourced to or performed by one or more third parties. Furthermore, any reference to singular includes plural embodiments, and any reference to more than one component or step may include a singular embodiment or step.

This disclosure relates to mid-face aesthetic scales and related methods for evaluating fullness in a mid-face area of a human patient's face for example, prior to and/or after a treatment such as a dermal filler regimen, a mid-face augmentation, or any other mid-face modification treatment (e.g., implants, surgical procedures, etc.).

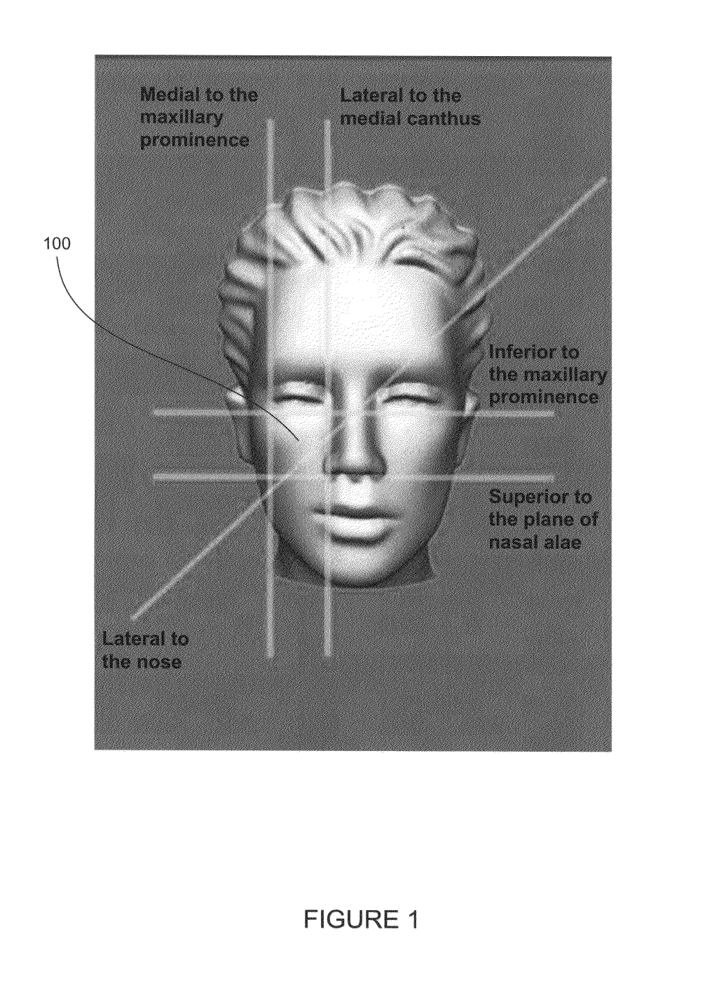

As used herein, with reference to FIG. 1, “mid-face” 100 is defined as a geometrically shaped area (e.g., triangle, pentagon, etc.) on either side of the face that is generally: (i) medial to the maxillary prominence, (ii) lateral to the medial canthus, (iii) inferior to the maxillary prominence, (iv) superior to the plane of nasal alae, and (v) lateral to the nose. The inventors have found that this definition of mid-face is best suited to focus medical professionals on appropriate malar anterior projection and mid-face fullness, and do so in a straightforward manner so that medical professionals can implement the methods described herein in a consistent manner.

An exemplary method for evaluating fullness in a mid-face area of a human patient's face comprises visually comparing a plurality of views of the patient's mid-face against a mid-face scale, and assigning a grade from the scale to the patient's mid-face based on the visual comparison.

An exemplary method for evaluating fullness in a mid-face area of a human patient's face comprises visually comparing (or having a trained medical professional visually compare) a plurality of views of the patient's mid-face against a 3 to 5 grade scale.

As used herein, a “medical professional” may be a medical doctor, doctor of osteopathic medicine, doctor of chiropractic, nurse, physician assistant, nurse practitioner, naturopathic medical doctor, licensed acupuncturist or the like.

As used herein, “human patients” include females and males, adults and children of all ages, and different ethnicities, for example, Caucasian, Hispanic, African-American, Asian, etc. In exemplary embodiments, patients are generally healthy and are seeking dermal filler regimens or other facial modification treatments primarily or purely for aesthetic reasons, whether to counter the aging process or to address general dissatisfaction with his/her appearance (e.g., an individual who is born with or later develops a relatively flat mid-face) or trauma. Stated differently, in exemplary embodiments, a human patient's face does not exhibit malar descent commonly associated with an illness or malady, e.g., HIV-associated facial lipoatrophy and the like.

In an exemplary embodiment, the scale is a three grade scale, from grade 1 representing a fairly full mid-face to grade 3 representing substantial loss of fullness in the mid-face area. In another exemplary embodiment, the scale is a four grade scale, from grade 1 representing a fairly full mid-face to grade 4 representing substantial loss of fullness in the mid-face area. In yet another exemplary embodiment, the scale is a five grade scale, from grade 1 representing a fairly full mid-face to grade 5 representing substantial loss of fullness in the mid-face area. As used herein, a “grade” is a term that merely refers to a classification within the scale, and is synonymous with terms like point, class, division, group, level, tier, etc. and the like.

While the various embodiments set forth herein have been described with reference to a 3 to 5 grade scale, persons skilled in the art will appreciate that this disclosure may be applied to scales having more or less grades depending on particular needs or circumstances.

In respect of the foregoing, persons skilled in the art will appreciate that the lowest or highest grade may correspond to a fairly full mid-face. Likewise, the lowest or highest grade may correspond to substantial loss of fullness in the mid-face area. Persons skilled in the art will also appreciate that, while the various grades may be designated by numbers, they may be designated by any alphanumeric character, symbol or the like, in some embodiments, as interpreted by a code.

In accordance with exemplary embodiments, a “view” is a medical professional's view of a human patient's mid-face area, either in person, in a photo or other digital media, or in a three-dimensional mold, the fullness of which is to be evaluated against a scale.

In accordance with exemplary embodiments, views of the patient's mid-face comprise one or more frontal mid-face views, and at least one mid-face view angled at between about 20 to about 50 degrees. In a preferred embodiment, two angled views of the patient's mid-face are compared to the scale, one from each of the right and left side of the patient's face. In yet other embodiments, a plurality of angled views of the patient's mid-face are compared to the scale.

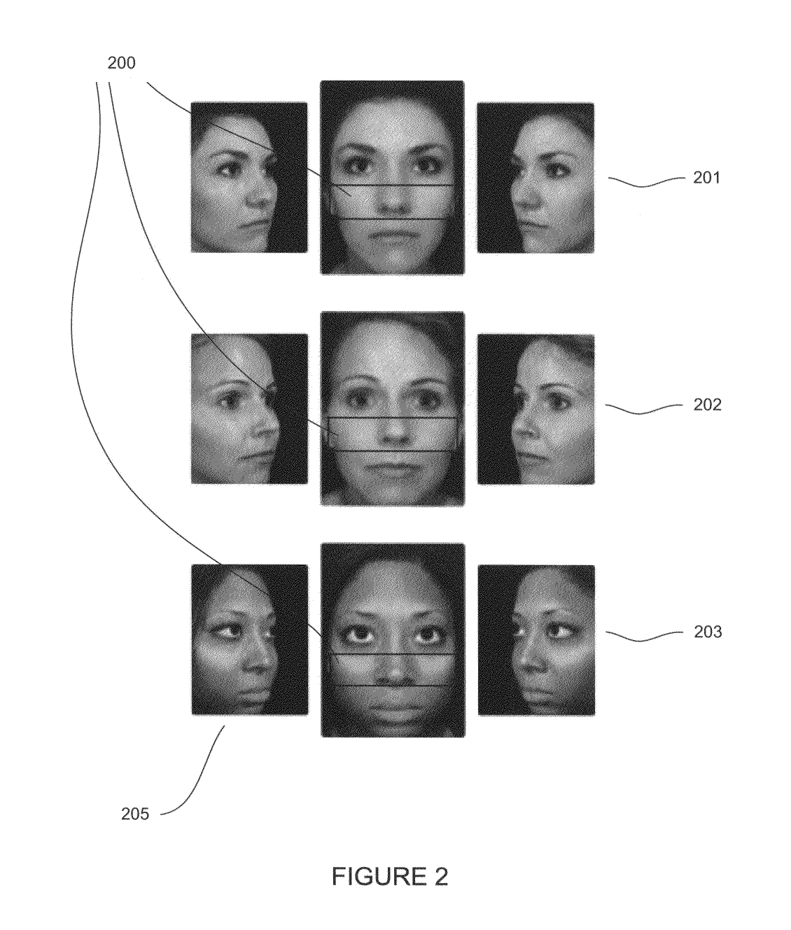

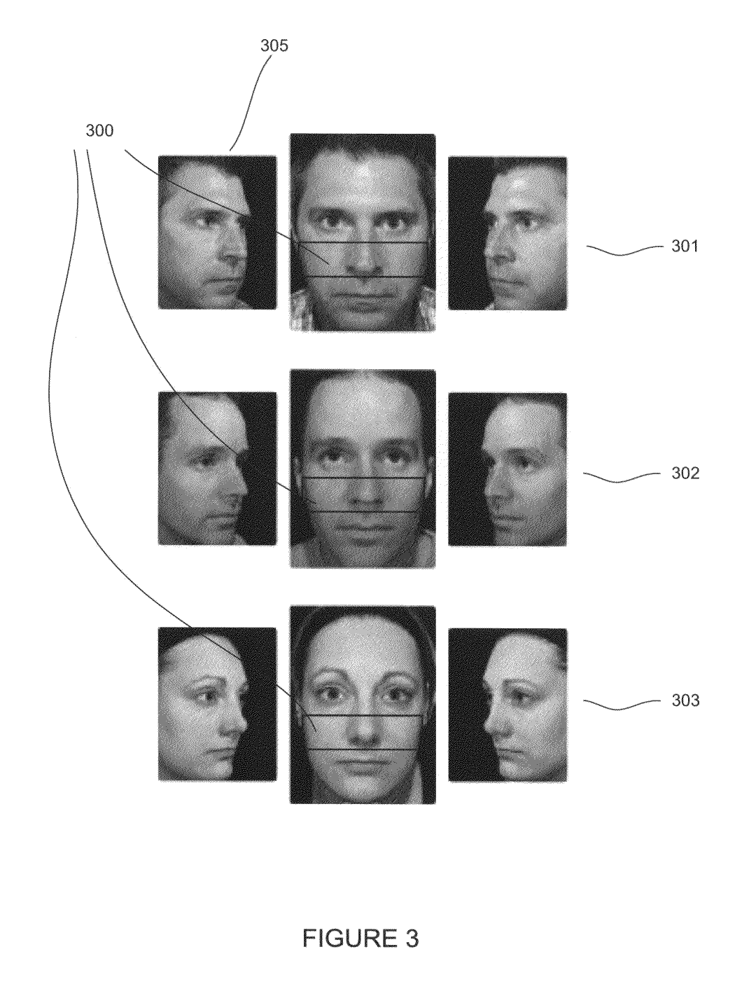

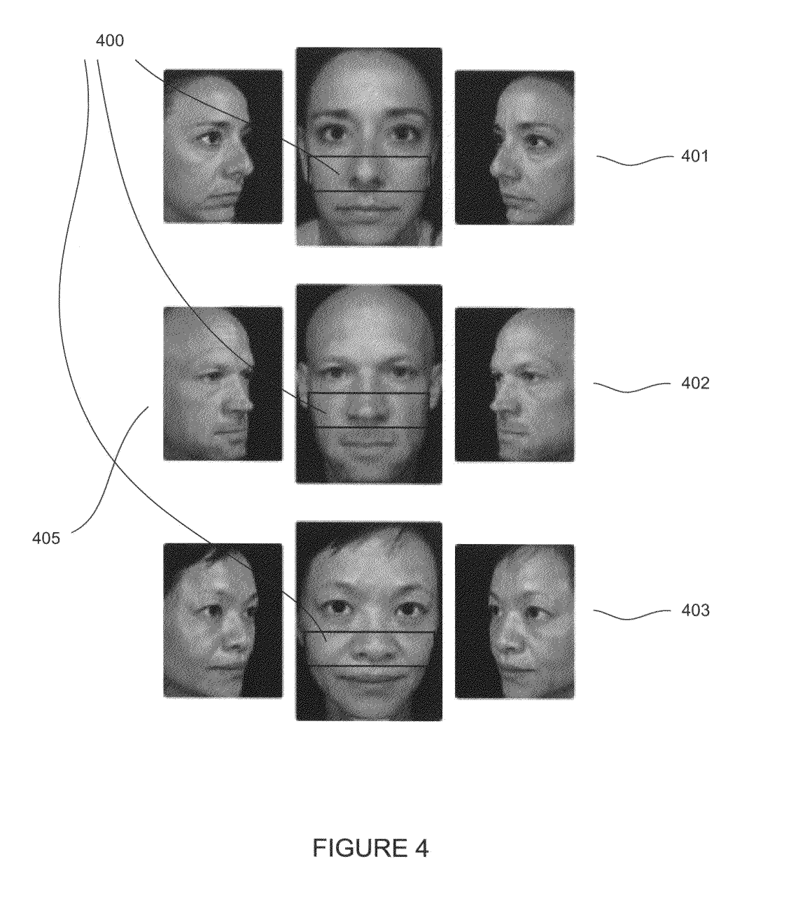

In accordance with exemplary embodiments, the mid-face views are angled within the range set forth above, for example, at about 22.5, 30 or about 45 degrees. In other embodiments, the angle is selected with reference to the ogee curve, or so that the tip of the nose “touches” the outer curve of the cheek in the view, as shown with reference to FIG. 3, by reference numeral 305. In yet other embodiments, the angle is selected so that the tip of the nose is inside the outer curve of the cheek in the view, as shown with reference to FIG. 2, by reference numeral 205. In still other embodiments, the angle is selected so that the tip of the nose is outside the outer curve of the cheek in the view, as shown with reference to FIG. 4, by reference numeral 405.

As described in further detail herein, selection of such an angle with the additional selection of appropriate lighting provides optimal observation of the actual mid-face fullness. However, in general, any angle that reveals the relative fullness of the mid-face, as that term has been defined by the inventors herein, is appropriate for use herein.

In accordance with exemplary embodiments, each grade of a 3 to 5 grade scale includes one or more image groupings of at least one reference subject's mid-face that illustrate a different fullness level of the mid-face area.

As used herein, a “reference subject” is a human whose mid-face is illustrative of a particular fullness level of the mid-face area or grade, typically without regard to his or her ethnicity, age, or gender.

In accordance with exemplary embodiments, the scale is presented using one, two, three, four, five or more reference subjects in each scale grade, more preferably two, three or four reference subjects, and most preferably three reference subjects in each scale grade.

In accordance with exemplary embodiments, different ethnicities, ages and/or genders are represented in the reference subjects, and within with same scale.

In accordance with exemplary embodiments, an “image” is a photograph or illustration of a reference subject's mid-face, whether in color or black and white, whether in a tangible or digital format, to be shown in a scale.

In accordance with exemplary embodiments, images of at least one reference subject's mid-face that illustrate a different fullness level of the mid-face area comprise one or more standard frontal mid-face images for each reference subject, and at least one standard mid-face image angled at between about 20 to about 50 degrees. In accordance with exemplary embodiments, the angle of the angled image(s) is “corresponding,” in that it is substantially similar or equivalent to the angle of the patient's mid-face angled view(s), as described above. By way of non-limiting example, if the angle of the patient's mid-face angled view(s) is 45 degrees, the angle of the angled image(s) may also be 45 degrees.

In a preferred embodiment, two angled images of the reference subject's mid-face are shown in the scale, one from each of the right and left side of the face of each reference subject. In yet other embodiments, a plurality of angled images of the reference subject's mid-face are shown in the scale.

In accordance with exemplary embodiments, the corresponding standard mid-face images are angled at about 22.5, 30 or about 45 degrees. As noted above, in other embodiments, the angle is selected with reference to the ogee curve, or so that the tip of the nose “touches” the outer curve of the cheek in the image. Again, any angle that reveals the relative fullness of the mid-face, as that term has been defined by the inventors herein, is appropriate for use herein.

In exemplary embodiments, images are arranged horizontally, vertically, diagonally, or otherwise, for example in an overlapping or tiled layout. In exemplary embodiments, images are grouped by reference subject and/or by grade of the 3 to 5 grade scale. In exemplary embodiments, all images are displayed together on a single tangible medium, for example, as a poster or chart. In other exemplary embodiments, only images illustrative of a single grade are displayed together on a single medium, while in yet other embodiments, images of a single reference subject are displayed alone, for example, as a flashcard. Persons skilled in the art will appreciate that, in addition to tangible mediums, images may be displayed on a computer monitor, point of interaction device (personal digital assistant (such as an iPhone® or Blackberry®), cellular phone, kiosk, etc.), a projection device, or the like.

In accordance with a preferred embodiment, the scale includes one or more images of three different reference subjects illustrative of each grade of a 4 grade scale, wherein the images of each reference subject are arranged horizontally, wherein the reference subjects are arranged vertically within each grade, wherein the grades are arranged horizontally, and wherein all images are displayed together on a single medium as a poster.

In accordance with exemplary embodiments, the scale includes no more than 80 images, more preferably no more than 60 images, and more preferably no more than 40 images. In accordance with a preferred embodiment, the scale includes 36 images.

In accordance with exemplary embodiments, the plurality of images comprises photographs of actual human subjects.

For the avoidance of doubt, the mid-face aesthetic scales described herein may be used in connection with any of the methods described herein, including without limitation, methods for baseline assessment of the mid-face region and/or for assessment of the post-treatment effectiveness of a facial treatment for improving the aesthetic appearance of the mid-face region; methods used as the primary efficacy measurement for mid-face augmentation in clinical studies; and methods used as a communication tool with patients to understand baseline, deficits, and possible and realized improvements associated with a facial treatment.

With reference to FIG. 2, mid-face focus region 200 of reference subjects 201, 202 and 203, each illustrating a grade 1 fullness level, may be evidenced by a fairly full mid-face. A grade 1 fullness level, may further be evidenced by cheek prominence projected beyond the infra-orbital rim at 45 degree view.

With reference to FIG. 3, mid-face focus region 300 of reference subjects 301, 302 and 303, each illustrating a grade 2 fullness level, may be evidenced by mild loss of fullness in mid-face area, flatness of mid-face, and/or cheek prominence at or behind infra-orbital rim. Exemplary reference subjects may have slight presence of tear trough but not extending past mid-eye. Exemplary reference subjects may also begin to show minimum volume loss of anterior cheek.

With reference to FIG. 4, mid-face focus region 400 of reference subjects 401, 402 and 403, each illustrating a grade 3 fullness level, may be evidenced by moderate loss of fullness with slight hollowing below malar prominence. A grade 3 fullness level, may further be evidenced by presence of the nasojugal groove extending past mid-eye.

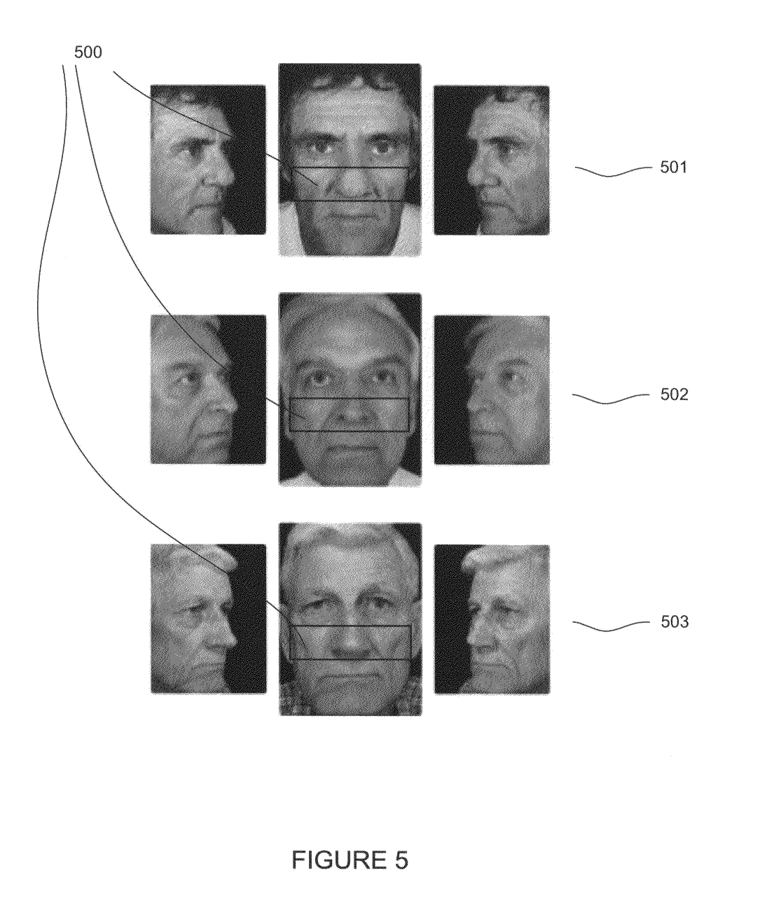

With reference to FIG. 5, mid-face focus region 500 of reference subjects 501, 502 and 503, each illustrating a grade 4 fullness level, may be evidenced by substantial loss of fullness in the mid-face area, clearly apparent hollowing below malar prominence. A grade 4 fullness level may further be evidenced by significant indentation in the mid-face area.

In exemplary embodiments, medical professionals are trained to recognize various characterizing features such as those noted herein, as well as others, of each grade of a 3 to 5 grade scale.

In accordance with exemplary embodiments and as mentioned briefly above, the plurality of images is captured with camera and light settings such that the concavity of the mid-face area is shown accurately and clearly. In accordance with an aspect of exemplary embodiments, auxiliary lighting or a flash is positioned above a reference subject in capturing an image, so as to take advantage of shadowing and thereby show malar anterior projection and mid-face fullness as if under normal lighting conditions. In accordance with an aspect of exemplary embodiments, auxiliary lighting or a flash is positioned from about 15 to about 60 degrees above the reference subject.

While various camera and light configurations could be implemented to achieve the foregoing results, an exemplary configuration comprises a camera system and an auxiliary flash unit.

An exemplary camera system in turn may comprise a high definition, digital camera with a wide angle lens (e.g., 28 mm) and built-in flash, such as the Canon G10. For people with light skin, an aperture of f5.0 and shutter speed of 1/125 second may be used. For people with medium skin, an aperture of f4.5 and shutter speed of 1/125 second may be used. For people with dark skin, an aperture of f3.5 and shutter speed of 1/125 second may be used. However, aperture and shutter speed will depend on a variety of external factors, and any aperture or shutter speed may be used that shows malar anterior projection and mid-face fullness as if under normal lighting conditions.

The camera may be centered between two reflectors, the camera's built-in flash and two reflectors together creating a soft overall illumination of the subject. The camera and reflectors may be coupled to an articulating arm that can rotate from 0 degrees to 90 degrees left and right around a pivot point. In an exemplary embodiment, the pivot point is located below a 9″ chin cup used to position subjects consistently. The camera may be tethered to a laptop computer that triggers the shutter and flash and displays the captured photograph onscreen. In an exemplary embodiment, multiple images can be displayed for comparison purposes and camera settings are saved along with subject data. The camera system can be set up on an elevated surface (e.g., a 30″ tabletop).

In an exemplary embodiment, an auxiliary flash unit (e.g., the Nikon Speed Light SB 800, having flash settings 1/32+⅔ EV) is positioned on a tripod directly behind and above the camera (e.g., 30″ above) at the 0 degree position and remains at the 0 degree position even when the angled photographs are taken. Stated differently, the auxiliary flash unit is aimed straight ahead as opposed to being aimed at the subject. The auxiliary flash unit may be configured to wirelessly synchronize with the camera's built-in flash when it detects the camera's flash. In an exemplary embodiment, a string is attached to the auxiliary flash unit to measure a consistent distance from the auxiliary flash unit to the subject. The auxiliary flash unit may be battery powered or is plugged into a standard electrical outlet. In an exemplary embodiment, the auxiliary flash provides the high-angle directional light that defines facial contours.

Other steps may be taken to ensure the concavity of the mid-face area is shown accurately and clearly, for example, pulling hair back away from face, removing make up, jewelry and piercings, instructing patients to look directly into the camera (as other eye positions may affect facial contours), maintain a “relaxed face” with no expression and remain stationary, and turning room light off and taking photographs in a darkened room.

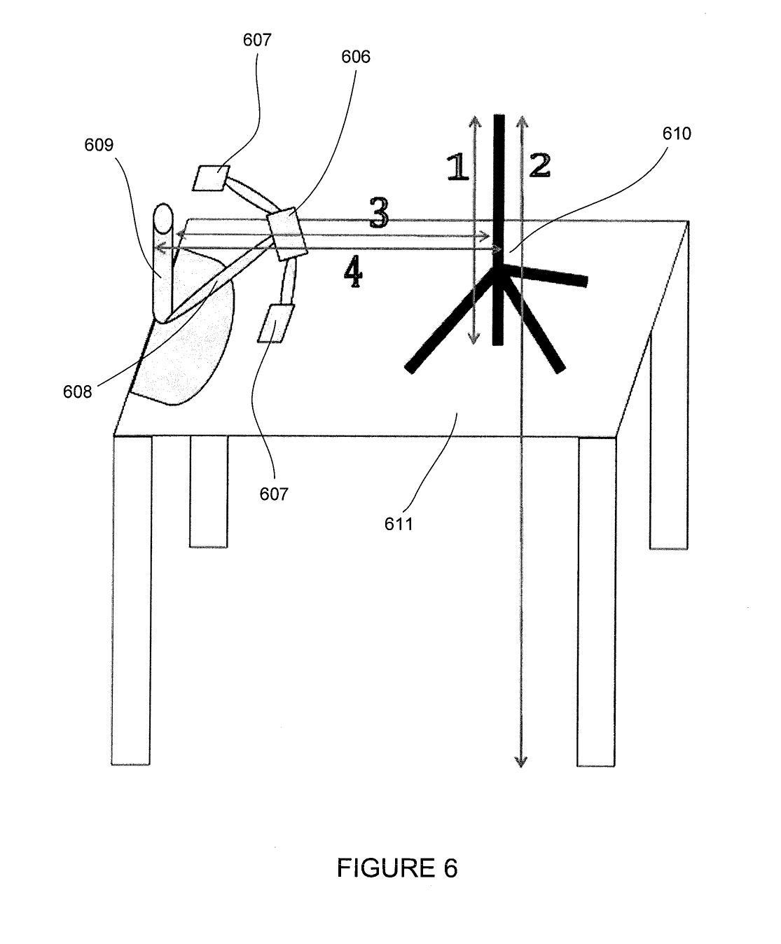

An exemplary camera system and an auxiliary flash unit, and with reference to FIG. 6, comprises a camera 606 centered between two reflectors 607, an articulating arm 608, a chin cup 609, and a tripod 610 for attachment of an auxiliary flash (not shown), all on an elevated surface 611.

In the embodiment shown, chin cup 609 and the center rod of tripod 610 are aligned approximately in a straight line to avoid uneven shadowing/light on either side of the subject's face, and distance 1 is measured from the top of tripod 610 (not including the height of the auxiliary flash) to elevated surface 611, and is approximately 29.5 inches; distance 2 is measured from the top of tripod 610 (not including the height of the auxiliary flash) to the floor, and is approximately 59 inches; distance 3 is measured from the inside edge of the center rod of tripod 610 to the inside edge of chin cup 609, and is approximately 34.5 inches; and distance 4 is measured from the outside edge of the center rod of tripod 610 to the outside edge of chin cup 609, and is approximately 32.75 inches.

In accordance with exemplary embodiments, the scale described herein is validated. In accordance with a preferred embodiment, the scale is validated regarding within- and between-observer agreement. As used herein, the “within-observer agreement” (aka intra-evaluator agreement) refers to the ability of each observer to reproduce their original score at a subsequent time, having allowed reasonable amount of time to elapse so that memory is not a likely factor. The “between-observer agreement” (aka inter-evaluator agreement) on the other hand, is the degree to which the observers will independently give a subject an identical score. In accordance with a preferred embodiment, weighted kappa coefficients are calculated to determine the statistical significance of the within-observer and between-observer agreement.

For both the within- and between-observer agreement the weighted kappa coefficients can be interpreted as follows:

0-0.19=Poor Agreement

0.20-0.39=Fair Agreement

0.40-0.59=Moderate Agreement

0.60-0.79=Substantial Agreement

0.80-1.0=Almost Perfect Agreement

In accordance with exemplary embodiments, the scale is validated using independent assessments by at least three, preferably at least four, evaluating investigators. In accordance with exemplary embodiments evaluating investigators rate at least 30, preferably at least 60, photographs, equally representing each grade of a 3 to 5 grade scale (e.g., 60 total photographs, 15 photographs representing each grade of a 4 grade scale), and do so on at least two occasions with at least 2 weeks between each occasion, wherein the same set of photographs are used at each occasion, but are presented to the evaluating investigators in a different order at each occasion.

In accordance with exemplary embodiments, the grades of a scale are validated by the within- and between-observer weighted kappa coefficients for the scale each being at least about 0.7, and optimally at least about 0.8.

As noted previously, an exemplary method for evaluating fullness in a mid-face area of a human patient's face comprises assigning (or having a trained medical professional assign) a grade of the scale to the patient's mid-face based on the visual comparison. The assigned grade may be charted by the medical professional or an assistant, and optionally conveyed to the patient.

In exemplary embodiments, the assigned grade is usable by medical professionals for assessing the fullness of the mid-face region, including use as a means for baseline assessment of the mid-face region and/or for assessment of the post-treatment effectiveness of dermal filler regimens or other facial modification treatments for improving the aesthetic appearance of the mid-face region (e.g., injection of HA-based products such as Perlane® or Perlane-L).

In other exemplary embodiments, the assigned grade is useful as the primary efficacy measurement for mid-face augmentation in clinical studies, for example, by researchers or administrative or regulatory bodies such as the United States FDA or other government authorities.

In yet other exemplary embodiments, the assigned grade is useful as a communication tool with patients to understand baseline, deficits, and possible and realized improvements associated with a dermal filler regimen or other facial modification treatment.

As noted above, an exemplary method for evaluating fullness in a mid-face area of a human patient's face may precede and/or follow a dermal filler regimen, a mid-face augmentation, or any other mid-face modification treatment (e.g., implants, surgical procedures, etc.).

Exemplary embodiments include a method of assessing the effectiveness of a dermal filler regimen on a mid-face area of a human patient's face comprising evaluating the mid-face area before delivery of the dermal filler and classifying it on a scale as disclosed above, delivering to the mid-face area an amount of the dermal filler, and assessing whether the dermal filler was effective by re-evaluating the mid-face area after delivery of the dermal filler.

In accordance with one aspect of an exemplary embodiment, a method of assessing the effectiveness of a dermal filler regimen further comprises re-delivering to the mid-face area an amount of the dermal filler if the mid-face area fails to show improvement on the scale after prior delivery of the dermal filler.

In accordance with another aspect of exemplary embodiments a method of assessing the effectiveness of a dermal filler regimen further comprises re-delivering an amount of the dermal filler if the mid-face area fails to show an at least 1 grade improvement on the scale after prior delivery of the dermal filler. In exemplary embodiments, re-delivering may occur approximately 1, 2, 3 or 4 weeks after prior delivery of the dermal filler, more preferably 2 weeks after.

In accordance with one aspect of an exemplary embodiment of this disclosure, a method of assessing the effectiveness of a dermal filler regimen further comprises assessing the effectiveness of a dermal filler regimen and assessing improvement in facial appearance in healthy human patients.

### EXAMPLE 1

A mid-face scale in accordance with an exemplary embodiment was developed and validated for grading mid-face fullness in a varied population. What follows is a description of the validation of a 4-point mid-face scale that can be used to grade mid-face fullness in clinical trials in support of mid-face augmentation as an indication for Perlane-L® Injectable Gel. It should be understood that the 4-point mid-face scale and validation methodology described forthwith are presented for purposes of illustration only and, while reflective of preferred aspects of the invention, should not be viewed as limiting the full scope of the invention.

Objectives

The validation study evaluated a 4-point mid-face scale regarding within- and between-observer agreement. Within-observer agreement refers to the ability of each observer to reproduce their original score at a subsequent time, having allowed reasonable amount of time to elapse so that memory is not a likely factor. Between-observer agreement is the degree to which the observers will independently give a subject an identical score.

Validation Procedure

The validation study included 60 subjects, assessed independently by four evaluating investigators. For each subject, three photos (front, right, and left sides) were provided to display a full view of the face. Each evaluator rated the subject's right and left mid-face for severity of volume loss or mid-face contour deficiency using the 4-point mid-face scale described in Table 1 below. The subjects used were selected to reflect the range of the scale. Each photograph had an ID-number, and the photos were arranged in different sequences for the first and second reviews. Assessments were made at two occasions with at least 2 weeks between those occasions. The same set of photographs were used at both occasions, but were presented to the evaluating investigators in a different order at each occasion, based on random assignment.

TABLE 1
Definition of mid-face scale (applied
to each side of the face separately)

| Grade | mid-face scale |
|---|---|
| 1 | Fairly full mid-face. |
| 2 | Mild loss of fullness in mid-face area. |
| 3 | Moderate loss of fullness with slight hollowing below |
| | malar prominence. |
| 4 | Substantial loss of fullness in the mid-face area, clearly |
| | apparent hollowing below malar prominaence. |

Each score in the mid-face scale was exemplified by the photo images on the scale. None of the photographs by which the scale was exemplified were used in the sets of photographs tested.

In the described validation study, each subject was randomly assigned two sequence numbers. These sequence numbers were used to create the order in which the subjects were presented for each round of review. The photos were then compiled into different booklets for each round of the validation review. The validation review booklets only contained the sequence numbers and not the subject identifier.

Each evaluating investigator received the mid-face scale (including exemplifying photographs), the set of photographs to be graded, and case report forms (CRFs). The assessments were made individually and the results were recorded in the CRFs. The assessments were not discussed between the Investigators.

The evaluating investigators provided a curriculum vitae or equivalent. The curriculum vitae contained the name, address and place of work, training, or other information that confirmed the suitability of the evaluating investigator responsible for the efficacy evaluations.

All data collected in the described validation study were recorded on the study CRFs. The results were entered in a spreadsheet, double checked, and the spreadsheet was sent to the statistician. Completed and signed original CRFs were collected by the end of each validation session. The original CRFs were sent to the sponsor.

Statistical Methods

Within-Observer Agreement

In the presently described validation study, four investigators evaluated each of the photographs at two occasions based on the 4-point mid-face scale rating scale. The agreement of these ratings within each investigator across the two occasions were assessed using the following two measures:

1. The overall proportion of the observed agreement, i.e. the sum of the number of observations in the main diagonal of the square matrix, divided by the total number of observations; and

2. An overall weighted kappa coefficient and corresponding 95% confidence interval, stratified by Investigator.

The within-observer agreement was also assessed for each investigator separately.

TABLE 2
Example of data presentation (combined
result from all 4 Investigators)

| | Session 2 | | | | |
|---|---|---|---|---|---|
| Session 1 | 1 | 2 | 3 | 4 | Total |
| 1 | x | x | x | x | x |
| 2 | x | x | x | x | x |
| 3 | x | x | x | x | x |
| 4 | x | x | x | x | x |
| Total | x | x | x | x | x |
| Exact Agreement | | | | | xx% |
| Weighted Kappa | | | | | x.xxx |
| (95% CI) | | | | | (x.xx, x.xx) |

Between-Observer Agreement

In the presently described validation study, four investigators evaluated each of the photographs at two occasions based on the 4-point mid-face scale rating scale. The agreement among the rating for the investigators was assessed for each of the six pairs of Investigators using the following two measures:

1. The overall proportion of the observed agreement between each pair, i.e. the sum of the number of observations in the main diagonal of the square matrix, divided by the total number of observations; and

2. A weighted kappa coefficient and corresponding 95% confidence interval for each pair, stratified by session.

TABLE 3
Example of data presentation

Investigator 2

| Investigator 1 | 1 | 2 | 3 | 4 | Total |
|---|---|---|---|---|---|
| 1 | | | | | |
| 2 | | | | | |
| 3 | | | | | |
| 4 | | | | | |
| Total | | | | | |
| Exact Agreement | | | | | xx% |
| Weighted Kappa | | | | | x.xxx |
| (95% CI) | | | | | (x.xx, x.xx) |

For both the within- and between-observer agreement the weighted kappa coefficients can be interpreted as follows:

0-0.19=Poor Agreement

0.20-0.39=Fair Agreement

0.40-0.59=Moderate Agreement

0.60-0.79=Substantial Agreement

0.80-1.0=Almost Perfect Agreement

The sample size chosen for the validation study is not based on any statistical arguments.

Additional analyses of within- and between-observer agreement were conducted within the demographic subgroups of race.

Results from Photographic Validation

Study Subjects

Photographs were used for all assessments.

Table 4 summarizes the demographic characteristics of the subjects used to photographically exemplify the mid-face volume for this validation. A total of 60 subject photographs were used to illustrate mid-face volume.

The mean age of the subjects was 45.7 years, with an age range of 23 to 80 years. More than half of the subjects (55%) were 35 years of age or older. The majority of subjects are female (72%) and Caucasian (87%); the remaining subjects are male (28%) and non-Caucasian (14%).

TABLE 4
Demographic Characteristics

| | mid-face scale |
|---|---|
| Parameter | (N = 60) |
| Age (years) | |
| n | 55 |
| Mean | 45.7 |
| SD | 16.34 |
| Median | 50.00 |
| Minimum | 23 |
| Maximum | 80 |
| Age Group n (%) | |
| 18-34 Years | 22 (37%) |
| 35-54 Years | 16 (27%) |
| ≧55 Years | 17 (28%) |
| Gender n (%) | |
| Female | 43 (72%) |
| Male | 17 (28%) |
| Race/Ethnicity N (%) | |
| Caucasian | 52 (87%) |
| Hispanic | 3 (5%) |
| African-American | 1 (2%) |
| Asian | 4 (7%) |

Within-Observer (Intra-Rater) Reliability

Assessments were made by each of the evaluators at two occasions (Round 1 and Round 2), at least 2 weeks apart. The same set of photographs was used for both rounds, but they were provided to the evaluators in a different order at each time. The agreement between the ratings of the same observer at the two separate rounds was the indicator of intra-rater reliability.

Right Side

The overall exact agreement was 87.1% between the Round 1 and Round 2 measurements for the right side. The overall within-observer weighted kappa value stratified by rater was 0.918, indicating almost perfect agreement within raters. The within-observer weighted kappa values varied between 0.816 and 0.954 among the different raters (see Table 5).

TABLE 5
Intra-Rater Reliability - Right Side

| Agreement between | | Weighted |
|---|---|---|
| Round 1 versus | Exact | Kappa |
| Round 2* | Agreement | (95% CI) |
| Rater 1 | 76.7% | 0.816 |
| | | (0.728, 0.903) |
| Rater 2 | 93.3% | 0.943 |
| | | (0.888, 0.997) |
| Rater 3 | 95.0% | 0.954 |
| | | (0.903, 1.005) |
| Rater 4 | 83.3% | 0.865 |
| | | (0.788, 0.943) |
| Overall* | 87.1% | 0.918 |
| | | (0.887, 0.949) |

*Weighted Kappa stratified by rater.

Left Side

The overall exact agreement was 86.3% between the Round 1 and Round 2 measurements for the left side. The overall within-observer weighted kappa value stratified by rater was 0.911, indicating almost perfect agreement within raters. The within-observer weighted kappa values varied between 0.813 and 0.954 among the different raters (see Table 6).

TABLE 6
Intra-Rater Reliability - Left Side

| Agreement between | | Weighted |
|---|---|---|
| Round 1 versus | Exact | Kappa |
| Round 2* | Agreement | (95% CI) |
| Rater 1 | 76.7% | 0.813 |
| | | (0.725, 0.902) |
| Rater 2 | 91.7% | 0.929 |
| | | (0.869, 0.989) |
| Rater 3 | 95.0% | 0.954 |
| | | (0.903, 1.005) |
| Rater 4 | 81.7% | 0.851 |
| | | (0.771, 0.932) |
| Overall* | 86.3% | 0.911 |
| | | (0.878, 0.943) |

*Weighted Kappa stratified by rater.

Within-Observer (Intra-Rater) Reliability by Race

Right Side

For the Caucasian subgroup, the exact agreement for all raters was 85.6% between the Round 1 and Round 2 measurements for the right side. The within-observer weighted kappa value was 0.904, indicating almost perfect agreement for all raters.

For the non-Caucasian subgroup, the exact agreement for all raters was 96.9% between the Round 1 and Round 2 measurements for the right side. The within-observer weighted kappa value was 0.907, indicating almost perfect agreement for all raters (see Table 7).

Exact agreement for all raters for the right side was slightly higher for non-Caucasian versus Caucasian (96.9% and 85.6%, respectively); however the weight kappa value was similar and both groups indicate almost perfect agreement.

TABLE 7
Intra-Rater Reliability by Race - Right Side

| Agreement | Caucasian | | Non-Caucasian | |
|---|---|---|---|---|
| between | | Weighted | | Weighted |
| Round 1 versus | Exact | Kappa | Exact | Kappa |
| Round 2 | Agreement | (95% CI) | Agreement | (95% CI)* |
| All Raters | 85.6% | 0.904 | 96.9% | 0.907 |
| | | (0.868, | | (0.730, |
| | | 0.940) | | 1.084) |

*Weighted Kappa stratified by rater.

Left Side

For the Caucasian subgroup analysis, the exact agreement for all raters was 84.6% between the Round 1 and Round 2 measurements for the left side. The within-observer weighted kappa value was 0.896, indicating almost perfect agreement for all raters.

For the non-Caucasian subgroup analysis, the exact agreement for all raters was 96.9% between the Round 1 and Round 2 measurements for the left side. The within-observer weighted kappa value was 0.907, indicating almost perfect agreement for all raters. See Table 8.

Exact agreement for all raters for the left side was slightly higher for non-Caucasian versus Caucasian (96.9% and 84.6%, respectively); however the weighted kappa value was similar and both groups indicate almost perfect agreement.

TABLE 8
Intra-Rater Reliability by Race - Left Side

| Agreement | Caucasian | | Non-Caucasian | |
|---|---|---|---|---|
| between | | Weighted | | Weighted |
| Round 1 versus | Exact | Kappa | Exact | Kappa |
| Round 2 | Agreement | (95% CI) | Agreement | (95% CI)* |
| All Raters | 84.6% | 0.896 | 96.9% | 0.907 |
| | | (0.859, | | (0.730, |
| | | 0.933) | | 1.084) |

*Weighted Kappa stratified by rater.

The within-observer weighted kappa values for the Caucasian and non-Caucasian subgroups were similar for both the right (0.904 and 0.907, respectively) and left sides (0.896 and 0.907, respectively) of the mid-face, and demonstrate almost perfect agreement for all raters.

Between-Observer (Inter-Rater) Reliability

The overall proportion of the observed agreement was calculated for the six pairs of evaluators, separately for the right and left mid-face sides.

Right Side

The exact agreement between the six pairs of raters varied between 72.5% and 87.5%, and the between-observer weighted kappa values varied between 0.759 and 0.893, indicating substantial to almost perfect agreement between raters (see Table 9).

TABLE 9
Inter-Rater Reliability - Right Side

| Agreement | Exact | Weighted Kappa* |
|---|---|---|
| between the raters | Agreement | (95% CI) |
| Rater 1 versus 2 | 76.7% | 0.811 |
| | | (0.748, 0.873) |
| Rater 1 versus 3 | 72.5% | .0759 |
| | | (0.689, 0.830) |
| Rater 1 versus 4 | 78.3% | 0.835 |
| | | (0.776, 0.893) |
| Rater 2 versus 3 | 87.5% | 0.893 |
| | | (0.842, 0.944) |
| Rater 2 versus 4 | 78.3% | 0.820 |
| | | (0.758, 0.881) |
| Rater 3 versus 4 | 77.5% | 0.808 |
| | | (0.745, 0.872) |
| Range across Pairs | 72.5-87.5% | 0.759, 0.893 |

*Weighted Kappa stratified by round of review

Left Side

The exact agreement between the six pairs of raters varied between 72.5% and 86.7%, and the between-observer weighted kappa values varied between 0.758 and 0.888 indicating substantial to almost perfect agreement between raters (see Table 10).

TABLE 10
Inter-Rater Reliability - Left Side

| Agreement | Exact | Weighted Kappa* |
|---|---|---|
| between the raters | Agreement | (95% CI) |
| Rater 1 versus 2 | 77.5% | 0.818 |
| | | (0.756, 0.879) |
| Rater 1 versus 3 | 72.5% | 0.758 |
| | | (0.686, 0.829) |
| Rater 1 versus 4 | 75.8% | 0.812 |
| | | (0.750, 0.873) |
| Rater 2 versus 3 | 86.7% | 0.888 |
| | | (0.836, 0.940) |
| Rater 2 versus 4 | 78.3% | 0.821 |
| | | (0.759, 0.882) |
| Rater 3 versus 4 | 76.7% | 0.801 |
| | | (0.737, 0.866) |
| Range across Pairs | 72.5-86.7% | 0.758-0.888 |

*Weighted Kappa stratified by round of review

Between-Observer (Inter-Rater) Reliability by Race

Right Side

For the Caucasian subgroup, the exact agreement between the six pairs of raters for the right side varied between 74.0% and 89.4%, and the between-observer weighted kappa values varied between 0.761 and 0.910 indicating substantial to almost perfect agreement between raters.

For the non-Caucasian subgroup, the exact agreement between the six pairs of raters for the right side varied between 68.8% and 100%, and the between-observer observer weighted kappa values varied between 0.763 and 1.000 indicating substantial to almost perfect agreement between raters. See Table 11.

TABLE 11
Inter-Rater Reliability by Race - Right Side

| | Caucasian | | Non-Caucasian | |
|---|---|---|---|---|
| Agreement | Exact | Weighted | Exact | Weighted |
| between the | Agree- | Kappa* | Agree- | Kappa* |
| raters | ment | (95% CI) | ment | (95% CI) |
| Rater 1 | 74.0% | 0.786 | 93.8% | 0.907 |
| versus 2 | | (0.716, 0.856) | | (0.730, 1.084) |
| Rater 1 | 73.1% | 0.761 | 68.8% | 0.763 |
| versus 3 | | (0.684, 0.838) | | (0.596, 0.931) |
| Rater 1 | 76.0% | 0.818 | 93.8% | 0.907 |
| versus 4 | | (0.753, 0.882) | | (0.730, 1.084) |
| Rater 2 | 89.4% | 0.910 | 75.0% | 0.800 |
| versus 3 | | (0.858, 0.962) | | (0.647, 0.953) |
| Rater 2 | 75.0% | 0.786 | 100% | N/A |
| versus 4 | | (0.715, 0.857) | | |
| Rater 3 | 77.9% | 0.809 | 75.0% | 0.800 |
| versus 4 | | (0.740, 0.878) | | (0.647, 0.953) |
| Range across | 74.0- | 0.761-0.910 | 68.8- | 0.763-1.000 |
| Pairs | 89.4% | | 100% | |

*Weighted Kappa stratified by round of review

Left Side

For the Caucasian subgroup, the exact agreement between the six pairs of raters for the left side varied between 73.1% and 88.5%, and the between-observer weighted kappa values varied between 0.759 and 0.906 indicating substantial to almost perfect agreement between raters.

For the non-Caucasian subgroup, the exact agreement between the six pairs of raters for the left side varied between 68.8% and 100%, and the between-observer observer weighted kappa values varied between 0.763 and 1.000 indicating substantial to almost perfect agreement between raters. See Table 12.

TABLE 12
Inter-Rater Reliability by Race - Left Side

| | Caucasian | | Non-Caucasian | |
|---|---|---|---|---|
| Agreement | Exact | Weighted | Exact | Weighted |
| between the | Agree- | Kappa* | Agree- | Kappa* |
| raters | ment | (95% CI) | ment | (95% CI) |
| Rater 1 | 75.0% | 0.795 | 93.8% | 0.907 |
| versus 2 | | (0.725, 0.864) | | (0.730, 1.084) |
| Rater 1 | 73.1% | 0.759 | 68.8% | 0.763 |
| versus 3 | | (0.681, 0.837) | | (0.596, 0.931) |
| Rater 1 | 73.1% | 0.790 | 93.8% | 0.907 |
| versus 4 | | (0.721. 0.858) | | (0.730, 1.084) |
| Rater 2 | 88.5% | 0.906 | 75.0% | 0.800 |
| versus 3 | | (0.853, 0.958) | | (0.647, 0.953) |
| Rater 2 | 75.0% | 0.787 | 100% | N/A |
| versus 4 | | (0.716, 0.857) | | |
| Rater 3 | 76.9% | 0.801 | 75.0% | 0.800 |
| versus 4 | | (0.730, 0.872) | | (0.647, 0.953) |
| Range across | 73.1- | 0.759-0.906 | 68.8- | 0.763-1.000 |
| Pairs | 88.5% | | 100% | |

*Weighted Kappa stratified by round of review

The between-observer weighted kappa values for the Caucasian and non-Caucasian subgroups were similar for both the right side (ranging from 0.761-0.910, and 0.763-1.000, respectively) and left side (ranging from 0.759-0.096, and 0.763-1.000, respectively) of the mid-face, indicating substantial to almost perfect agreement across the pairs of raters.

Discussion

The validation study evaluated a 4-point mid-face scale regarding within (intra)- and between (inter)-evaluator agreement for the right and left sides of the mid-face. A total of 60 subjects were evaluated in Round 1 and Round 2 of the validation. Diverse age groups, genders, and ethnicities were represented in the subjects used to photographically evaluate the mid-face scale in order to evaluate mid-face fullness in a varied population.

The intra-observer agreement (ability of each evaluator to reproduce their original score at a subsequent time) was evaluated using weighted kappa coefficients interpreted by associated categorical grading. The overall within-observer weighted kappa value stratified by rater was 0.918 for the right side and 0.911 for the left side. Both scores indicated almost perfect agreement within the 4 raters for their ability to independently provide an identical score for the same subject during two temporally discrete occasions. The overall exact agreement was consistent for both the right and left sides (87.1% and 86.3%, respectively).

The variation of weighted kappa coefficients for between-observer agreement was consistent between the right and left side, with agreement scores ranging from 0.759 to 0.893 for the right side, and 0.758 to 0.888 for the left side, indicating substantial to almost perfect agreement between raters for each side of the mid-face scale.

Subgroup analysis by race demonstrates similarity of the weighted kappa values for the within- and between-observer reliability for both the right and left sides of the mid-face, and provides further evidence that the mid-face scale is a valid assessment tool for evaluating mid-face volume loss and/or contour deficiency for people of different racial backgrounds.

Based on the results of intra- and inter-observer ratings using weighted kappa coefficients, it was concluded that the 4-point mid-face scale is considered suitable for use in clinical trials to grade mid-face fullness.

### EXAMPLE 2

A clinical trial was conducted using an exemplary mid-face scale as the measurement tool and tested to evaluate whether the scale could be used to measure changes created using dermal fillers for mid-face augmentation and if the changes were clinical meaningful. Forty subjects were enrolled in the study at two sites. The mean age of the subjects was 53.1 years and the majority of the subjects (85%) were females. This population is very consistent with the general population seeking mid-face augmentation.

The primary objective was to identify whether Perlane-L® was effective in the treatment of mid-face volume loss and/or mid-face contour deficiency as determined by the blinded evaluator's assessment using the mid-face scale at week 8 after the mid-face augmentation compared to the baseline condition. A responder was defined as a subject showing at least a one-grade improvement on the mid-face scale on the both the right and lefts sides of the face from the baseline condition. This study also assessed the percentage of subjects who had at least one grade improvement using the commonly accepted Global Aesthetic Improvement Scale (“GAIS”) by the subject, the treating physicians and the blinded evaluator.

All of the subjects had a moderate to severe grade on the mid-face scale at the baseline of the study. Results of the primary effectiveness analysis showed that 97.5% ( 39/40) of the subjects were responders at week 8. The results of GAIS at week 8 were comparable to those measured in mid-face scale: 100% of the subjects were assessed as having improved from baseline by both the blinded evaluator and the treating physician, and 95% of the subjects judged that their appearance improved from baseline using GAIS.

While GAIS is not an objective measurement tool nor is it a validated tool, GAIS improvement has been accepted by the medical community as clinical meaningful, visible aesthetic results. The almost perfect correlation between a one-grade increase and an assessment of at least “improved” on the GAIS strongly indicates that a one-grade improvement in grade on the mid-face scale represents clinical meaningful aesthetic results that are visible to medical professionals (e.g., the treating physicians and blinded evaluators) as well as to non-professionals (e.g., the study subjects).

While the various embodiments set forth herein have been described with reference to dermal fillers, persons skilled in the art will appreciate that the methods may be applied equally to other mid-face modification treatments, such as a mid-face augmentation, or any other mid-face modification treatment (e.g., implants, surgical procedures, etc.) without departing from the spirit and scope of this disclosure.

Moreover, benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the invention. The scope of the invention is accordingly to be limited by nothing other than the appended claims, in which reference to an element in the singular is not intended to mean “one and only one” unless explicitly so stated, but rather “one or more.” Moreover, where a phrase similar to ‘at least one of A, B, or C’ or ‘at least one of A, B, and C’ are used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. All structural, chemical, and functional equivalents to the elements of the above-described exemplary embodiments that are known to those of ordinary skill in the art are expressly incorporated herein by reference and are intended to be encompassed by the present claims. Further, a list of elements does not include only those elements but may include other elements not expressly listed or inherent to such process, method, article, or apparatus.

We claim:

1. A method for evaluating fullness in a mid-face area of a human patient's face comprising:
visually comparing a plurality of patient views of the patient's mid-face against a mid-face scale;
wherein the mid-face is defined as medial to the maxillary prominence, lateral to the medial canthus, inferior to the maxillary prominence, superior to the plane of nasal alae, and lateral to the nose;
wherein the plurality of patient views comprises
a frontal mid-face view, and
a mid-face view angled at between about 20 to about 50 degrees from each of the right and left side of the patient's face; and
wherein the mid-face scale comprises three to five grades, each grade of the mid-face scale including at least one image grouping of at least one reference subject's mid-face, and each illustrating a different fullness level of the mid-face;
each image grouping further comprising
a standard frontal mid-face image for each reference subject, and
a corresponding standard mid-face image angled at between about 20 to about 50 degrees from each of the right and left side of the face of each reference subject; and
determining a patient grade on the mid-face scale based on the visual comparison.

2. The method according to claim 1, wherein the scale is a 4 grade scale.

3. The method according to claim 2, wherein the scale is presented using a plurality of reference subjects in each scale grade.

4. The method according to claim 3, wherein different ethnicities are represented in the reference subjects.

5. The method according to claim 3, wherein different ages are represented in the reference subjects.

6. The method according to claim 3, wherein different genders are represented in the reference subjects.

7. The method according to claim 3, wherein the scale includes no more than 60 images.

8. The method according to claim 3, wherein the plurality of images comprise photographs of actual human subjects.

9. The method according to claim 3, wherein the corresponding standard mid-face images are angled at about 45 degrees.

10. The method according to claim 3, wherein the plurality of images is captured with camera and light settings such that the concavity of the mid-face area is shown accurately and clearly.

11. The method according to claim 3, wherein the within- and between-observer weighted kappa coefficients for the scale are each at least about 0.7.

12. A method of assessing the effectiveness of a dermal filler regimen on a mid-face area of a human patient's face comprising:
evaluating the mid-face area before delivery of the dermal filler and classifying it on a 3 to 5 grade scale, wherein each grade of the scale includes a plurality of image groupings of at least one reference subject's mid-face that illustrate a different fullness level of the mid-face,
delivering to the mid-face area an amount of the dermal filler, and
assessing whether the dermal filler was effective by re-evaluating the mid-face area after delivery of the dermal filler.

13. The method of claim 12, wherein the method comprises re-delivering to the mid-face area an amount of the dermal filler if the mid-face area fails to show an at least 1 grade improvement on the scale after prior delivery of the dermal filler.

14. The method of claim 12, wherein the method is applied to assess improvement in facial appearance in healthy human patients.

15. A method for evaluating fullness in a mid-face area of a human patient's face comprising:
visually comparing a plurality of patient views of the patient's mid-face against a mid-face scale;
wherein the mid-face is defined as medial to the maxillary prominence, lateral to the medial canthus, inferior to the maxillary prominence, superior to the plane of nasal alae, and lateral to the nose;
wherein the mid-face scale comprises three to five grades; and
wherein the grades of the mid-face scale are validated by the between-observer weighted kappa coefficient for the scale being at least about 0.7; and
determining a patient grade on the mid-face scale based on the visual comparison.

16. The method of claim 15, wherein the grades of the mid-face scale are validated by the between-observer weighted kappa coefficient for the scale being at least about 0.8.

17. The method of claim 15, wherein the grades of the mid-face scale are validated by the within-observer weighted kappa coefficient for the scale being at least about 0.7.

18. The method of claim 15, wherein the within- and between-observer weighted kappa coefficients are calculated using independent assessments by at least three evaluating investigators.

19. The method of claim 15, wherein at least three evaluating investigators rate at least 30 photographs, equally representing each grade of the scale, and do so on at least two occasions with at least 2 weeks between each occasion.

20. The method of claim 19, wherein the same set of photographs are used at each occasion, but are presented to the evaluating investigators in a different order at each occasion.

21. A mid-face scale for evaluating fullness in a mid-face area of a human patient's face comprising:
from three to five grades, each grade of the mid-face scale including at least one image grouping of at least one reference subject's mid-face, and each illustrating a different fullness level of the mid-face, comprising
a frontal mid-face image for each reference subject, and
a mid-face image angled at between about 20 to about 50 degrees from each of the right and left side of the face of each reference subject,
wherein the mid-face is defined as medial to the maxillary prominence, lateral to the medial canthus, inferior to the maxillary prominence, superior to the plane of nasal alae, and lateral to the nose.

22. The mid-face scale of claim 21, wherein the plurality of image groupings of at least one reference subject's mid-face are arranged horizontally, wherein the reference subjects are arranged vertically within each grade, wherein the grades are arranged horizontally, and wherein all images are displayed together on a single medium as a poster.

23. The mid-face scale of claim 21, wherein the images are captured with a high definition camera and with auxiliary lighting or a flash is positioned from about 15 to about 60 degrees above the reference subjects.
